(19) United States
(12) Patent Application Publication
(10) Pub. No.: US 2012/0207862 A1
(43) Pub. Date: Aug. 16, 2012

(54) ORAL INHIBITORS OF AGE-RELATED NADH OXIDASE (arNOX), COMPOSITIONS AND NATURAL SOURCES

(76) Inventors: D. James Morré, West Lafayette, IN (US); Dorothy M. Morré, West Lafayette, IN (US); Thomas Shelton, Delray Beach, FL (US)

(21) Appl. No.: 13/224,317

(22) Filed: Sep. 1, 2011

Related U.S. Application Data
(60) Provisional application No. 61/379,564, filed on Sep. 2, 2010.

(51) Int. Cl. A61K 36/45; A61K 36/53; A61K 36/537; A61K 36/00

(52) U.S. Cl. 424/732; 424/725; 424/745; 424/746; 424/776

(57) ABSTRACT

Described are compositions and agents for blocking serum and other aging factors, especially arNOX in serum, skin or other body fluid or tissue and/or on the cell surface, and methods for using the same. More particularly, the invention relates to agents comprising any one of several naturally-occurring arNOX inhibitors capable of reducing occurrence or severity of or treating disorders and complications of disorders resulting from cell damage caused by aging-related isoforms of NADH oxidase (arNOX). In one exemplary embodiment, nutraceutical, cosmeceutical or pharmaceutical compositions comprise at least one naturally occurring arNOX inhibitor or inhibitor source. Such naturally occurring inhibitors also are capable of augmenting the anti-arNOX effect of other naturally occurring arNOX inhibitory agents. The discovery of multiple inhibitors with significantly different kinetics of inhibition that when combined provide long term arNOX inhibition is one of the unique and non-obvious features of the present methods and compositions.

### CROSS-REFERENCE TO RELATED APPLICATIONS

This application claims priority to U.S. Provisional Patent Application No. 61/379,564, filed on Sep. 2, 2010, which is hereby incorporated by reference in its entirety to the extent there is no inconsistency with the present disclosure.

### BACKGROUND

This disclosure relates to reducing the incidence or severity of aging-related disorders caused by oxidative damage by an aging-specific isoform of NADH oxidase (arNOX) and to treatment of aging-related disorders caused by oxidative damage by arNOX using oral dietary supplements, certain pharmaceutical or topical applications, specific combinations of derivatives of benzoic acid and water soluble flavonoids and/or herbal sources providing such substances some of which are employed as culinary seasonings and/or infusions or extracts of such herbal sources effective as low dose arNOX inhibitors in clinical trials.

Cell surface proteins with hydroquinone (NADH) oxidase activity (designated NOX) that function as terminal oxidases of plasma membrane electron transport to complete an electron transport chain involving a cytosolic hydroquinone reductase, plasma membrane located quinones and the NOX proteins have been described, at least in part (Kishi et al., 1999, Biochem. Biophys. Acta 1412:66-77 and Morré, 1998, Plasma Membrane Redox Systems and their Role in Biological Stress and Disease, Kluwer Academic Publishers, Dordrecht, NL, pp. 121-156). This system provides a rational basis for operation of the mitochondrial theory of aging and for propagation of aging related mitochondrial lesions, including a decline in mitochondrial ATP synthetic capacity and other energy-dependent processes during aging (Boffoli et al., 1996, Biochem. Biophys. Acta 1226:73-82; Lenaz et al., 1998, BioFactors 8:195-204; de Grey, 1997, BioEssays 19:161-166; and de Grey, 1998, J. Anti-Aging Med. 1:53-66).

The plasma membrane NADH oxidases, NOX or ENOX proteins are unique to the cell surface and exhibit both hydroquinone (NADH) oxidase and protein disulfide-thiol interchange activities that normally respond to hormone and growth factors. arNOX (or ENOX3) proteins are a family of growth-related proteins that are associated uniquely with aging cells and are generally hormone unresponsive.

The aging-related isoform of NADH oxidase (arNOX) is a member of the ENOX3 family of ENOX proteins. The circulating form of arNOX increases markedly in human sera and in lymphocytes of individuals after the age of 30. The arNOX protein is uniquely characterized by an ability to generate superoxide radicals, which may contribute significantly to aging-related changes including, but not limited to, oxidation of skin proteins, atherogenesis and other action-at-a-distance aging phenomena. Activity of arNOX in aging cells and in sera has been described previously (Morré and Morré, 2006, Rejuvenation Res. 9:231-236). See also WO 2011/022387.

Aging has been proposed to result from an ever-increasing level of destructive chemical reactions involving free radicals, with mitochondria as the principal mediators of the process (Harman, 1956, J. Gerontol. 11:298-300 and Harman, 1972, J. Am. Geriatr. Soc. 20:145-147). The main line of reasoning to support these ideas is that, of all subcellular components, mitochondria are both a major source of free radicals and a major direct victim of free radical damage. As a result, loss of mitochondrial function may be the driving intracellular change underlying aging, and the cause of other pro-oxidant changes such as slower protein turnover. There is considerable indirect as well as direct experimental support for the theory. For example, a decline in ATP synthesis capacity and of energy-dependent processes during aging has been reported (Syrovy and Gutmann, 1997, Exp. Gerontol. 12:31-35; Sugiyama et al., 1993, Biochem. Mol. Biol. Intl. 30:937-944; Boffoli et al., 1996, Biochim. Biophys. Acta 1226:73-82; and Lenaz et al., 1998, BioFactors 8:195-204).

Age and oxidative stress are major risk factors for heart disease (Schmuck et al. 1995. Clin. Chem. 41:1628-1632). A large body of evidence supports the notion that reactive oxygen species provide a causal link in the appearance of oxidized circulating lipoproteins such as oxidized LDLs and their subsequent clearance by macrophages and delivery to the arterial wall. It now appears likely that oxidized LDL is a major contributor to progressive atherogenesis by enhancing endothelial injury, by inducing foam cell (lipoprotein engorged macrophages) generation and associated smooth muscle proliferation (Holvoet et al. 1999. Ther. Apher. 3:287-293). Macrophages clear the circulation of oxidized lipoprotein particles by internalizing them and in so doing are transformed into foam cells. The foam cells deliver their cargo of oxidized fats and cholesterol where they are deposited beneath the arterial wall. Such progressive delivery of oxidatively-damaged lipoprotein particles eventually leads to atherosclerotic plaques and advanced heart disease.

However, the basis for LDL oxidation has been little studied. Levels of common antioxidants including α-tocopherol, β-carotene and ascorbate decline with age but there is no apparent correlation between ingestion of these common antioxidants and amelioration of the aging process or decreased mortality (Bjelakovic 2007. JAMA 297:842-857). The implication is that the oxidative damage leading to aging and increased atherogenic risk is the result of a much more specific causation.

Why does LDL oxidation increase in the elderly and why is it greater in some individuals than in others? Our findings suggest that LDL oxidation in the elderly and in individuals at high risk for heart disease correlates with levels of circulating arNOX. The arNOX proteins are shed into the milieu surrounding the cells. In aged individuals, the amount of superoxide generated by the shed arNOX proteins has been measured to be quite substantial reaching a maximum at age 65 to 75 in males and age 55-65 in females. Of those who die of a heart attack, 85% are 65 or older (American Heart Association Statistical Update. Heart disease and stroke statistics—2008. Circulation 17:e25-e146). Women surviving beyond age 65 usually have diminished arNOX levels compared to men and a lower risk of cardiovascular disease compared to men (Kannel et al. 2003. Progress in Cardiovascular Nursing 18:135-140) further suggesting some causal relationship between arNOX levels and atherogenic risk.

This model of the effects of arNOX is consistent with the Mitochondrial Theory of Aging, which holds that during aging, increased reactive oxygen species in mitochondria cause mutations in the mitochondrial DNA and damage mitochondrial components, resulting in senescence. The Mitochondrial Theory of Aging proposes that accumulation of spontaneous somatic mutations of mitochondrial DNA (mtDNA) leads to errors of mtDNA-encoded polypeptide chains (Manczak M et al., 2005, J. Neurochem. 92(3):494-504). These errors, occurring in mtDNA-encoded polypeptide chains, are stochastic and randomly transmitted during mitochondrial and cell division. The consequence of these alterations is defective oxidative phosphorylation. Respiratory chain defects may become associated with increased oxidative stress amplifying the original damage (Ozawa, 1995, Biochim. Biophys. Acta 1271:177-189; and Lenaz, 1998, Biochim. Biophys. Acta 1366:53-67). In this view, therefore, mutated mitochondrial DNA, despite being present only in very small quantities in the body, may be the ultimate major cause of oxidative stress.

Where accumulation of somatic mutations of mtDNA leads to defective oxidative phosphorylation, a plasma membrane oxido-reductase (PMOR) system has been suggested to augment survival of mitochondrially deficient cells through regeneration of oxidized pyridine nucleotide (de Grey, 1997, BioEssays 19:161-166; de Grey, 1998, Anti-Aging Med. 1:53-66; Yoneda et al., 1995, Biochem. Biophys. Res. Comm. 209:723-729; Schon et al., 1996, Cellular Aging and Cell Death, Wiley and Sons, New York, pp. 19-34; Ozawa et al., 1997, Physiol. Rev. 77:425-464; and Lenaz, 1998, BioFactors 8:195-204). However, alterations of mtDNA of themselves have been difficult to link to other forms of cellular and tissue changes related to aging. Chief among these is low density lipoprotein (LDL) oxidation and atherogenesis (Steinberg, 1997, J. Biol. Chem. 272:20963-20966) and oxidation of skin proteins (Morré et al., 2010, Rejuvenation Res. 13:162-164; Morré et al., 2010, J. Invest. Dermatol., Submitted).

A model to link accumulation of lesions in mtDNA to extracellular responses was first proposed with rho⁰ cells (Larm et al., 1994, J. Biol. Chem. 269:30097-30100; Lawen et al., 1994, Mol. Aspects. Med. 15:s13-s27; de Grey, 1997, BioEssays 19:161-166; and de Grey, 1998, Anti-Aging Med. 1:53-66). Similar studies have been conducted with transformed human cells in culture (Vaillant et al., 1996, Bioenerg. Biomemb. 28:531-540).

Under conditions where plasma membrane oxidoreductase (PMOR) is overexpressed, electrons are transferred from NADH to external acceptors by a defined electron transport chain, resulting in the generation of reactive oxygen species (ROS) at the cell surface. Such cell surface-generated ROS may then propagate an aging cascade originating in mitochondria to both adjacent cells as well as to circulating blood components such as low density lipoproteins (Morré and Morré, 2006, Rejuvenation Res. 9:231-236) and interstitial fluids surrounding skin collagen and elastin (Kern et al., 2010, Rejuvenation Res. 13:165-167).

In view of physical and quality of life issues associated with aging and further in view of the aging of the general population, there is a need in the art for agents that reduce the deleterious ability of arNOX to generate reactive oxygen species (ROS) for the purposes of reducing or treating the resultant physiological conditions, such as oxidation of lipids and proteins in low density lipoprotein particles (LDLs) and attendant arterial changes and to maintain skin vitality (Morré and Morré, 2006, Rejuvenation Res. 9:231-236; Morré et al., 2010, Rejuvenation Res. 13:162-164).

The arNOX activity of aging cells has been shown to be inhibited by naturally occurring agents such as coenzyme Q (ubiquinone) including CoQ₁₀, CoQ₉ and CoQ₈ (Morré et al., 2008, BioFactors 32:231-235). However, the use of coenzyme Q is not completely satisfactory for several reasons: it is costly, it oxidizes easily with a concomitant loss of efficacy, and preparations containing coenzyme Q must be specially packaged to prevent loss of function during storage. Thus, while some currently available antioxidant preparations and methods might inhibit arNOX activity, challenges still exist. Accordingly, there is a need for improvements in the art to augment or even replace previously disclosed agents and techniques with the agents and techniques that inhibit arNOX but are also non-toxic and naturally occurring.

### DEFINITIONS

As used herein, the term “disorder” refers to an ailment, disease, illness, clinical condition, or pathological condition.

As used herein, the term “reactive oxygen species” refers to oxygen derivatives from oxygen metabolism or the transfer of free electrons, resulting in the formation of free radicals (e.g., superoxides or hydroxyl radicals).

As used herein, the term “antioxidant” refers to compounds that neutralize the activity of reactive oxygen species or inhibit the cellular damage done by said reactive species.

As used herein, the term “pharmaceutically acceptable carrier” refers to a carrier medium that does not interfere with the effectiveness of the biological activity of the active ingredient, is chemically inert, and is not toxic to the patient to whom it is administered.

As used herein, the term “pharmaceutically acceptable derivative” refers to any homolog, analog, or fragment which exhibits arNOX inhibitory activity and is relatively non-toxic to the patient to whom it is administered.

“Age-related NADH oxidase” (arNOX) refers to a cell surface aging-related NADH enzyme, which can be measured as described herein. See also WO 2011/022387.

In the present context a nutritional supplement refers to a composition comprising one or preferably more than one inhibitor of arNOX that is in liquid form, or formulated as a pill, tablet, or capsule to be taken at least once a day, so as to provide an effective dose for inhibiting arNOX, and thus, ameliorating or diminishing the effects of aging in a human or animal to whom the effective dose is administered. Alternatively, the active ingredients (arNX inhibitors) can be formulated as a functional food, such as a cookie, bar or cereal, for example.

### SUMMARY OF THE INVENTION

The present disclosure provides compositions and methods for inhibiting serum aging-related factors (specifically the aging-related isoform of NADH oxidase, abbreviated arNOX herein), and methods for using the same, and more specifically, it relates to compositions comprising inhibitors of arNOX and combinations of arNOX inhibitors that are derivatives of benzoic acid or flavins alone, in combination or from natural sources, including certain culinary seasonings. These several naturally-occurring arNOX inhibitors alone, and especially in combinations, provide for a method to reduce arNOX-mediated damage or treat disorders and/or complications of disorders resulting from cell damage caused by (arNOX). In one embodiment, the arNOX inhibitory compositions comprise at least one naturally occurring arNOX inhibitor. In other embodiments, more than one, for example three, are combined to yield a sustained release formulation.

Other inhibitors of arNOX include tyrosol, hydroxytyrosol and coenzyme Q. The compounds can be formulated into drinks, powdered drinks, tablets, pills, capsules or in functional foods or other nutritional supplements such that taken at least once a day, a dose effective for inhibiting arNOX is provided to the human or animal consuming the formulated arNOX-inhibitory composition.

Provided herein are nutritional supplements and pharmaceutical compositions, methods of use, and pharmaceutical kits for reducing the severity of or for treating disorders resulting from oxidative changes in cells that result in aging by targeting an aging-related isoform of NADH oxidase (arNOX) shed into the sera by aging cells or reducing the harmful effects of arNOX action.

The present methods and compositions are based, at least in part, on the discovery that certain dietary constituents, often referred to as “Herbes de Provence,” inhibit the activity of at least one aging-related isoform of NADH oxidase (arNOX) shed into the sera by aging cells. Herbes de Provence typically comprise basil (Ocimum basilicum), summer savory (Satureja hortensis), oregano (Oreganum vulgare), thyme (Thymus vulgaris), and may further comprise one or more of lavender (Lavandula angustifolia), marjoram (Origanum majorana), rosemary (Rosmarinus officinalis), sage (Salvia officinalis), fennel seed (Foeniculum vulgare), with the ratio of component herbs varying with personal or regional choice. Tarragon (or estragon, dragon's-wort, Artemisia dracunculus) is also especially useful in the compositions and methods for anti-aging and arNOX inhibition herein. The inhibition of arNOX by these substances results in a decrease in the generation of reactive oxygen species by arNOX and may serve as a possible explanation for the “French Paradox” wherein the French diet and/or lifestyle leads to reduced atherogenic risk despite a diet rich in butter and cholesterol (Teissedre et al., 2000, J. Agric. Food Chem. 48:3801-3804; Morré et al., 2010, Rejuvenation Res. 13:165-167). Certain of these herbs also may have benefit by inhibiting platelet adhesion and aggregation (Yazdanparast and Shaphrivarv, 2008, Vascul. Pharmacol. 48:32-34). A decrease in reactive oxygen species is believed to cause a decrease in oxidative damage resulting from said reactive oxygen species generated via arNOX. There are methods for inhibiting cell membrane-associated arNOX and soluble arNOX in sera provided herein.

In the nutritional supplements provided herein, there are at least two herbal components selected from among basil (Ocimum basilicum), summer savory (Satureja hortensis), oregano (Oreganum vulgare) and thyme (Thymus vulgaris), and the nutritional supplements may further comprise one or more of lavender (Lavandula angustifolia), marjoram (Origanum majorana), Lamiaceae), rosemary (Rosmarinus officinalis), sage (Salvia officinalis) and fennel seed (Foeniculum vulgare), and/or tarragon (Artemisia dranunculus). The ratio of the dry weights of basil (Ocimum basilicum), summer savory (Satureja hortensis), oregano (Oreganum vulgare) and thyme (Thymus vulgaris) can be 1:1 to 10:1 of components from among summer savory (Satureja hortensis), oregano (Oreganum vulgare) and thyme (Thymus vulgaris, and/or tarragon (Artemisia dranunculus) or they may be in ratio(s) of 1:1 to 1:10 for basil, lavender, marjoram, fennel seed and sage.

Of the herbs listed basil, tarragon (especially French tarragon), rosemary, marjoram, sage and savory (especially summer savory) are particularly active as arNOX inhibitors. Accordingly, an herbal extract or infusion should comprise at least 10-100% of one or more of the foregoing, advantageously more than one of the foregoing.

In an herbal mixture, components can be incorporated in the following proportions: basil, 0-95%; thyme, 0-50%; oregano, 0-90%; tarragon, 0-95%; rosemary, 0-95%; lavender, 0-50%; sage, 0-95%; savory, 0-95%; marjoram, 0-95%, (each weight/weight); basil, 0-75%; thyme, 0-50%; oregano, 0-75%; tarragon, 0-75%; rosemary, 0-75%; lavender, 0-50%; sage, 0-75%; savory, 0-75%; marjoram, 0-75% (each weight/weight); or comprising basil, 0-30%; thyme, 0-30%; oregano, 0-30%; tarragon, 0-30%; rosemary, 0-30%; lavender, 0-30%; sage, 0-30%; savory, 0-30%; marjoram, 0-30% (each weight/weight).

Advantageously, at least two of the foregoing are incorporated in an herbal mixture useful as a nutritional supplement for inhibiting arNOX.

10-100% of an extract or infusion preparation for use in the present anti-aging compositions should be an extract and/or infusion of at least one of basil, tarragon (especially French tarragon), rosemary, marjoram, sage and savory (especially summer savory), which can be incorporated in a nutritional supplement or a cosmeceutical formulation for topical use. Similarly, an herbal nutritional supplement in dried form for oral ingestion should comprise one or more of basil, tarragon (especially French tarragon), rosemary, marjoram, sage and savory (especially summer savory), and advantageously two or more thereof.

A cosmeceutical preparation for inhibiting the deleterious effects of arNOX on appearance should be formulated using infusions, extracts or tinctures of at least one of basil, tarragon (especially French tarragon), rosemary, marjoram, sage and savory (especially summer savory). For topical application, the preparation may further comprise an emollient, astringent, moisturizer gel, cream, or other carrier suitable for applying to the skin, as well known to the art. Additional beneficial components may also be incorporated into such preparations, again as well known to the art. In addition, transdermal patches may be prepared using the ingredients described herein in combination with materials suitable for transdermal use, with the patches being shaped for such target areas as under or over the eyes, at the outside corner of the eyes, forehead, in the area between the eyebrows, nasolabial fold areas, neck, among others.

In any of the foregoing preparations, one or more components including one or more of an arNOX-inhibiting benzoic acid (or a pharmaceutically acceptable salt thereof) or gallic acid (or a pharmaceutically acceptable salt thereof), catechin, salicin, allopuranol or apigenin.

In another embodiment, there are methods and compositions for screening assays to identify agents that inhibit arNOX.

The pharmaceutical compositions, nutritional supplements and cosmeceutical compositions comprising compounds or materials that inhibit arNOX can be administered via various modes of administration. The modes of administration of compounds include, but are not limited to, oral, topical, mucosal or intradermal, subcutaneous, intravenous, intraperitoneal or intramuscular administration. Oral administration can be using capsules, tablets, soft gels, solutions or other ingestible format. The materials in the composition, including any excipients or carriers, must be at least food grade and should be accepted as safe for human consumption, although pharmaceutical grade materials may be used. For mucosal administration, there can be use of suppositories, aerosols or sprays, solutions or gels, and the components, including excipients or carriers, are advantageously of pharmaceutical grade. For administration via injection, it is understood that sterile and pharmaceutical grade materials are used. For topical administration, the arNOX inhibiting materials may be formulated with excipients or carriers appropriate to the site of use, including in the form of aerosol, emollient, moisturizing, astringent, lotion, cream, ointment, gel or other format suitable for topical use. Also encompassed herein are kits for administering arNOX-inhibiting materials to a person or animal to benefit from the administration thereof.

In various other exemplary embodiments, the composition further includes a cosmetically or pharmaceutically acceptable carrier. In some exemplary embodiments, the arNOX inhibitory agent is present together with other arNOX inhibitors derived from naturally occurring sources including, but not limited to, culinary herbs and/or extracts, infusions or tinctures thereof. In various exemplary embodiments, arNOX inhibitor agents from one source are augmented by the effects of arNOX inhibitory agents from another source.

One of ordinary skill in the art recognizes that the arNOX inhibitory compositions described herein can be administered in any convenient manner compatible with the material and the route of administration. In some embodiments, the compositions are formulated for oral administration in the form of liquids, gel capsules, tablets or sustained release granules. In these and other embodiments, the arNOX inhibitory agent is provided at a concentration of between 200 and 600 mg/capsule or tablet or other dosage form.

Infusions of herbs can be prepared by mixing about 100 to about 2000 mg, advantageously about 500 mg of dried herb(s) with 1 cup boiling water and allowed to steep for 5-10 minutes. Alternatively, the solutions are allowed to cool slowly at room temperature and steep 1-24 hours, advantageously 6-20, or 12-18 hours. Advantageously, at least two herbs, as described above, are included. A typical dose would be 1 or 2 cups daily.

The magnitude of a therapeutic dose of arNOX inhibitor in the acute or chronic management of aging-related oxidative damage varies with the severity of the condition to be treated and the route of administration. The dose and dose frequency also vary according to the age, body weight, condition and response of the individual patient, and the inhibitor source and/or combination of sources used. Importantly, the dosage for an individual should not result in discomfort or toxicity.

All combinations described herein are encompassed as therapeutic and it is understood that one of skill in the art can determine a proper dosage of particular inhibitor mixtures using the parameters provided herein. In general, the total daily dose ranges of the active materials for the conditions described herein are generally from about 10 mg to about 2000 mg administered in divided doses administered parenterally, mucosally, orally or topically. A preferred total daily dose is from about 200 mg to about 600 mg of a combination of herbs and/or natural products as described herein. In general, natural materials such as herbs are administered at a higher daily dosage than more concentrated materials, such as purified compounds, and also extracts, tinctures or infusions of natural materials such as Herbes de Provence.

In various embodiments of the present methods, the arNOX inhibitory capsules or tablets are taken orally at least once (or two times) daily. Advantageously, at least one dosage form is as a sustained release formulation. Preferably, the sustained release formulation is provided in a manner that maintains a constant level of inhibition for at least 12 hours of a 24 hour period.

These and other features and advantages of the present compositions and methods are set forth or will become more fully apparent in the description that follows and in the appended claims. The features and advantages may be realized and obtained by means of the formulations and combinations particularly pointed out in the appended claims and through the practice of the methods herein.

### BRIEF DESCRIPTION OF THE FIGURES

Various exemplary embodiments of the compositions and methods according to the invention will be described in detail, with reference to the following figures wherein:

FIG. 1 provides a graphical depiction of kinetic data that show that salicin is a competitive inhibitor of arNOX.

FIG. 2 provides a listing of competitive inhibitors of xanthine oxidase that also are inhibitors of arNOX.

FIG. 3 illustrates structure-function analyses of several known inhibitors of arNOX already used in topical applications to inhibit arNOX.

FIG. 4 provides a summary of active arNOX inhibitors resulting from screening based on structure-activity relationships of FIG. 3. Of 50 positives obtained, approximately 50% were derivatives of benzoic acid. In addition to salicin, previously known to inhibit, the listed compounds, along with gallic acid, are given as examples of active benzoic acid derivatives. A typical dose response is shown for gallic acid.

FIG. 5 shows natural sources of gallic acid (3,4,5-trihydroxybenzoic acid) equivalents (derivatives of benzoic acid such as gallic acid that occur naturally). One of the best sources was French seasonings such as savory.

FIG. 6 shows that pomegranate infusion, a rich natural source of gallic acid equivalents, was also extremely active in inhibiting arNOX activity in human saliva.

FIG. 7 shows that (±)-catechin, another constituent associated with French seasonings, inhibits arNOX whereas the substituted catechins found in green tea, for example, do not.

FIG. 8A-8B provide HPLC traces of infusions of two savory preparations from the same provider (McCormicks brand) with different expiration dates, one of which (FIG. 8A) was inactive in inhibiting arNOX and one of which (FIG. 8B) was active. Fraction 24, as shown in FIG. 8B, appeared to be present in greater abundance in the active savory preparation compared to fraction 20 in the inactive savory preparation to account for the arNOX inhibitory activity of the active preparation.

FIG. 9 illustrates the results of difference mass spectrometric analysis of Fraction 24 (difference between Fraction 24 (active) and Fraction 20 (inactive). The material at 623.0908 mass units decomposed to yield apigenin (270.9 mass units) as did the material at 453.1274 and the material at 437.1446 which differed by an —OH moiety. These compounds appeared to be water soluble derivatives of the flavonoid apigenin, conjugated with sugars or hydroxybenzoic acid. The material at 149.0638 mass units was a dimethylated benzoic acid.

FIG. 10 shows that apigenin aglycone supplied as a DMSO solution (insoluble in water) inhibited arNOX activity at 10 μM.

FIG. 11 shows the dose response of apigenin aglycone supplied in a DMSO solution.

FIG. 12 shows that blueberry tincture (prepared in ethanol) as another source of flavonoid compounds, inhibits arNOX activity. Blueberries or other blue fruits, fresh or frozen, are extracted with ethanol (125 mg/ml) to prepare a tincture which was assayed directly (60 μg/2.5 ml) or assay mixture. Ethanol alone was without effect.

FIG. 13 shows that various dimethylated benzoates (mass 149.0638), irrespective of the position of the two methyl substitutions, markedly inhibited arNOX activity in saliva.

FIG. 14 demonstrates that arNOX activity of saliva and sera correlate, thus the supporting the conclusion that inhibition of salivary arNOX activity translates directly into inhibition of serum arNOX activity.

FIG. 15 provides a time course of salivary arNOX activity of a 73 year old male subject after oral administration of a 1350 mg capsule of savory leaf and a 1350 mg capsule of estragon leaf (French tarragon) at time 0 h. Maximum inhibition was achieved after 1 h, followed by return to base line activity.

FIG. 16 shows the results of an experiment carried out as in FIG. 15, except that two 200 mg capsules of domestic tarragon were ingested at time 0 h. Maximum inhibition was achieved after 4 h, with return to base line activity after 8 h.

FIG. 17 shows the results of an experiment carried out as in FIG. 15, except that two 350 mg capsules of finely ground savory were administered orally at time 0 h. Maximum inhibition as achieved after 5 h, with return to base line activity after 8-9 h.

FIG. 18 shows the results of an experiment carried out as in FIG. 17, to show reproducibility of the response to finely ground savory administered at time 0 h. Maximum inhibition was achieved after 6 h, with return to base line activity after 12 h.

FIG. 19 shows the results of an experiment carried out as in FIG. 17, except there were orally administered two 350 mg of finely ground savory (as in FIG. 4) and two 200 mg capsules of savory leaf (as in FIG. 1) at time 0 h. Two inhibition maxima were observed; one at 2 h corresponding to savory leaf (FIG. 1) and one at 6 h corresponding to ground savory (see also FIG. 4). arNOX activity returned to base line after 16 h.

FIG. 20 shows the results of an experiment carried out as in FIG. 15, except that two X 300 mg of gallic acid were orally administered at time 0 h. Maximum inhibition (84%) was delayed until 8 h, with a return to base line activity between 11 and 14 h.

The present disclosure provides for oral nutritional supplements, including fortified drinks, pharmaceutical or cosmetic compositions, methods of use, and pharmaceutical and cosmetic (cosmeceutical) preparations for health and well-being benefits, including but not limited to, the reduction in severity of or treatment of disorders resulting from oxidative changes in cells that result in aging by targeting an aging-related isoform of NADH oxidase (arNOX), shed into the sera by aging cells. The compositions may contain natural materials such as fresh or dried plant material, other natural materials, or agents extracted from plants or chemically synthesized, or semisynthetic active materials. For example, the compositions described herein may comprise at least one extract, tincture or infusion or a purified compound shown to inhibit arNOX activity, whether alone or with other inhibitory agents that, at least partially, inhibit or block the activity of an aging-related isoform of NADH oxidase shed into the sera by aging cells. The composition may comprise natural material known to comprise active agents useful in inhibiting arNOX, or active extracts or agents derived therefrom, optionally together with other compounds known to the art. Such other compounds may comprise excipients and/or carriers, gums, fillers, preservatives and the like.

Progressive development of age-associated systematic disease has long been associated with production of reactive oxygen species (ROS) (Linnane et al. 2007. Biogerentology 8:445-467). Our work has focused on an age-related oxidase (arNOX) as an important source of ROS especially in the circulation. Since arNOX proteins are shed from the cell surface and circulate, they coexist with low density lipoproteins in the blood and appear to be responsible for their oxidation. Oxidized lipoproteins are endocytosed by macrophages to form the foam cells which are ultimately responsible for the deposition of cholesterol and oxidized lipids in the arterial walls as the basis for formation of fatty streaks leading to atherosclerotic plaques and coronary heart disease (Holvoet Pet al. 1998. Circulation 98:1487-1494; Holvoet et al. 2001. Anterioscler. Thromb. Vasc. Biol. 21:844-848.

As primary ROS generators, the arNOX proteins emerge as the major source of superoxide anion in the blood rather than mitochondria. Superoxide generation by arNOX molecules accounts for the observations that the bulk of the ROS in the blood are produced by the plasma membrane or in the circulation itself. Estimates suggest that several millimoles per ml of superoxide are generated daily in the near proximity of lipoproteins just from the circulating forms of arNOX.

Without wishing to be bound by any particular theory, it is believed that arNOX is responsible for LDL oxidation, which correlates with atherogenesis. arNOX inhibitors, for example, administered orally in the form of herbal nutritional supplements or in the form of infusions, extracts or tinctures, ameliorate and/or prevent or slow the progression of coronary artery disease. In particular the nutritional supplements can be based on various herbal seasonings, including but not limited to those known collectively as Herbes de Provence. In addition, combinations of herbal materials and infusions, extracts and/or tinctures can be used for health and well-being and cosmetic benefit.

Ingestion of such preparations results in decreased generation of reactive oxygen species by arNOX; this inhibition of arNOX provides a possible explanation for the French Paradox, wherein the French lifestyle leads to reduced atherogenic risk despite a cholesterol-rich diet high in cheese and butter. Previous studies attributed the reduction in risk to consumption of red wine as a natural source of the polyphenol resveratrol (Teissedre et al. 2000. J. Agric. Food Chem. 48:3801-3805). However, the herbs listed in the text, which are staples of the French diet, offer a more compelling explanation. Certain of these herbs and phenolics may have benefit as well by inhibiting platelet adhesion and aggregation (Yazdanparast and Shahriyary. 2008. Vascul Pharmacol 2008; 48:32-37).

### EXAMPLE 1

### Characterization of arNOX inhibitors

Reduction of ferric cytochrome c by superoxide was employed as a standard measure of superoxide formation (Mayo, L. A. and Curnutte, J., 1990, Meth. Enzyme. 186:567-575; Butler, J. et al., 1982, J. Biol. Chem. 257:10747-10750). This is a widely accepted method when coupled to superoxide dismutase inhibition for the measurement of superoxide generation. The assay consists of 150 μl buffy coats in PBSG buffer (8.06 g NaCl, 0.2 g KCl, 0.18 g Na₂HPO₄, 0.13 g CaCl₂, 0.1. g MgCl₂, 1.35 g glucose dissolved in 1000 ml deionized water, adjusted to pH 7.4, filtered and stored at 4° C.). Rates were determined using an SLM Aminco DW-2000 spectrophotometer (Milton Roy, Rochester, N.Y., USA) in the dual wave length mode of operation with continuous measurements over 1 min every 1.5 min. After 45 min, test compounds were added and the reaction was continued for an additional 45 min. After 45 min, a millimolar extinction coefficient of 19.1 cm⁻¹ was used for reduced ferricytochrome c. Extracts were made of the potentially inhibitory compounds in water unless otherwise indicated.

Protein amounts were determined by the bicinchoninic acid procedure (Smith et al. (1985) Anal. Biochem. 150:70-76).

### EXAMPLE 2

### Oral arNOX Inhibitor Screening Strategy

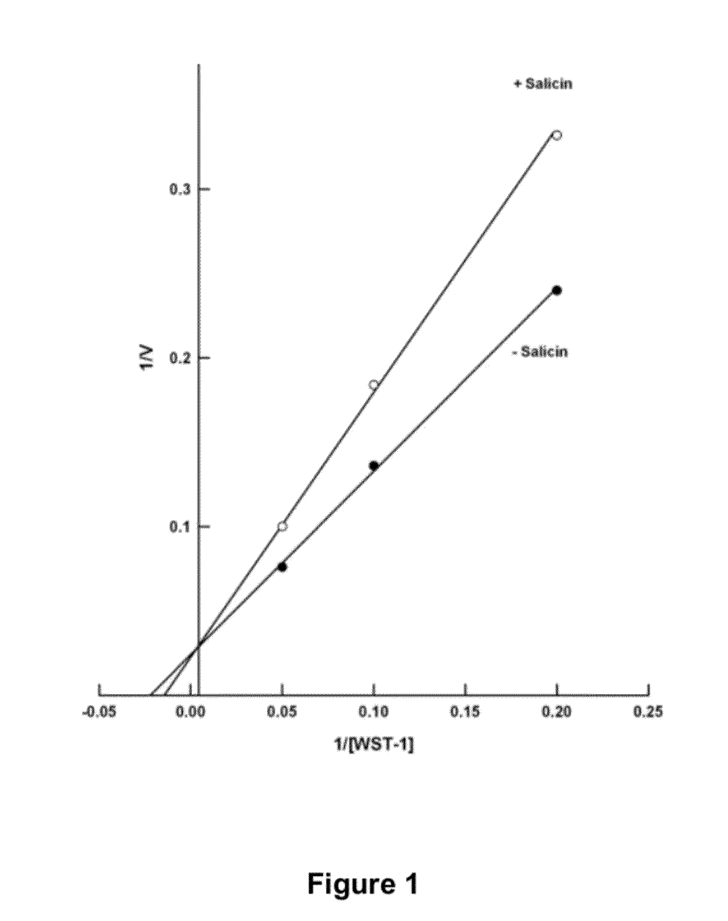

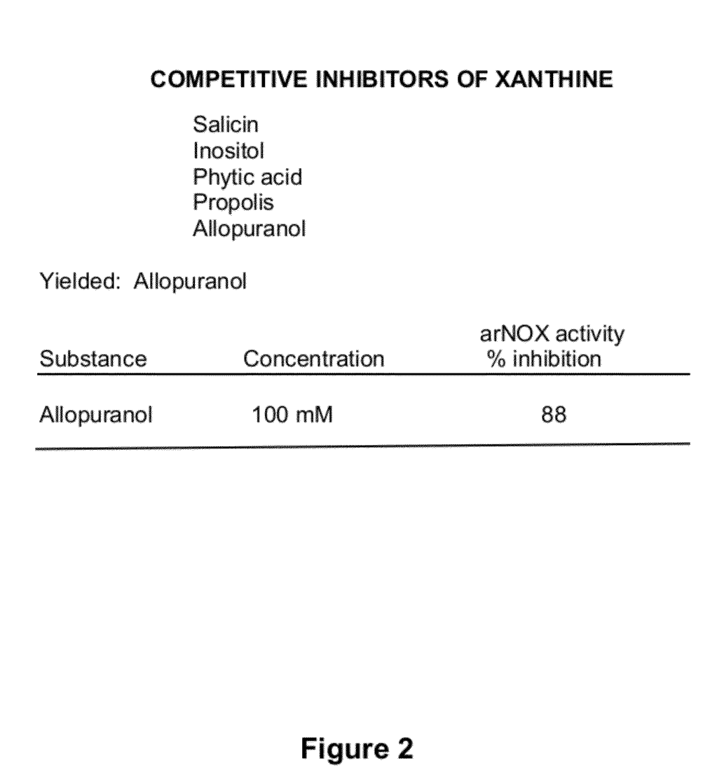

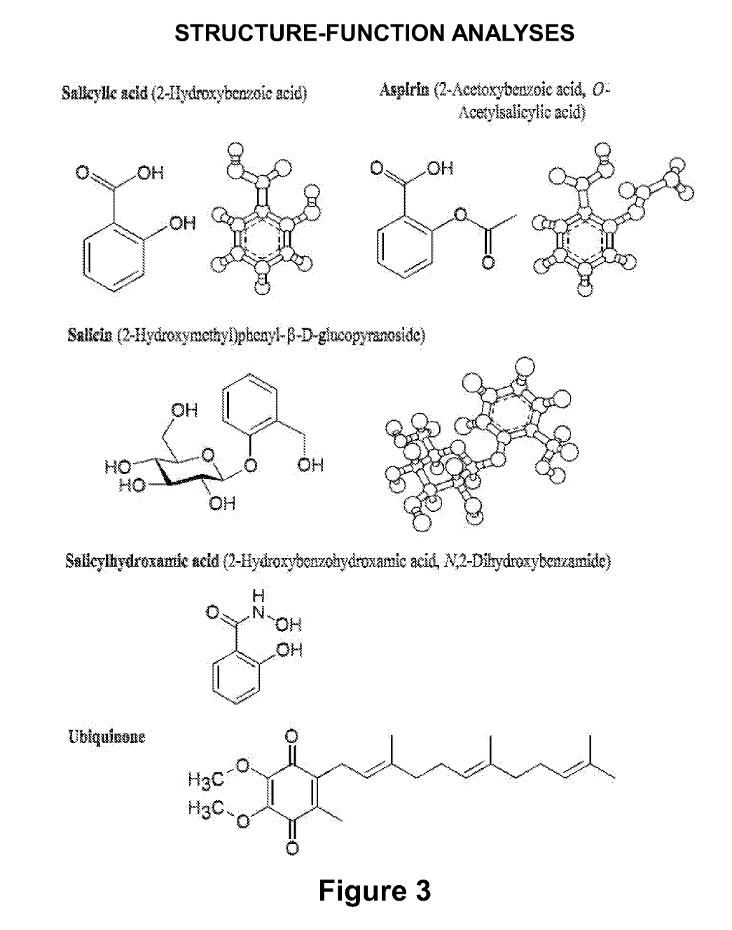

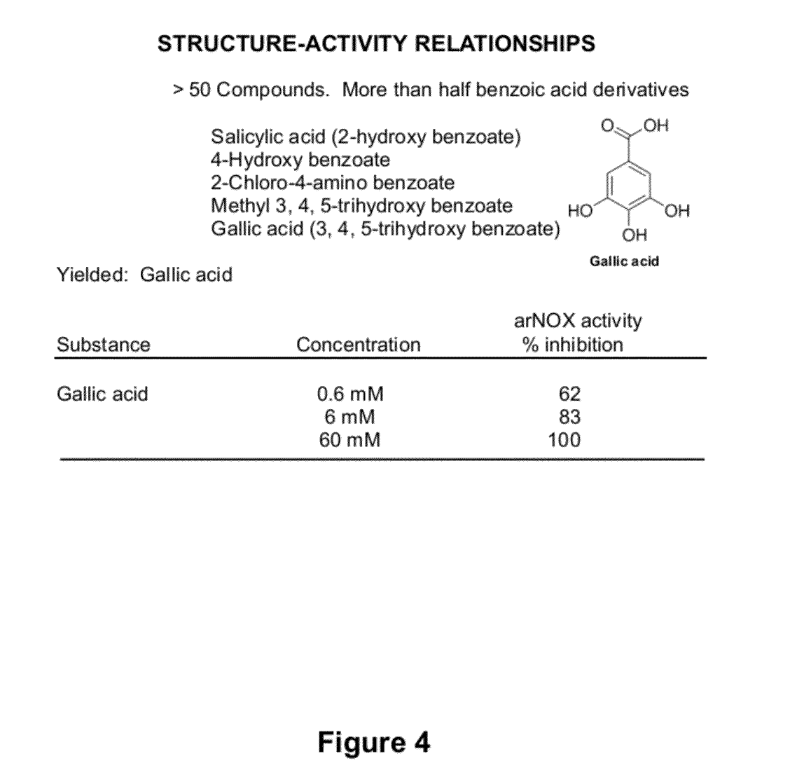

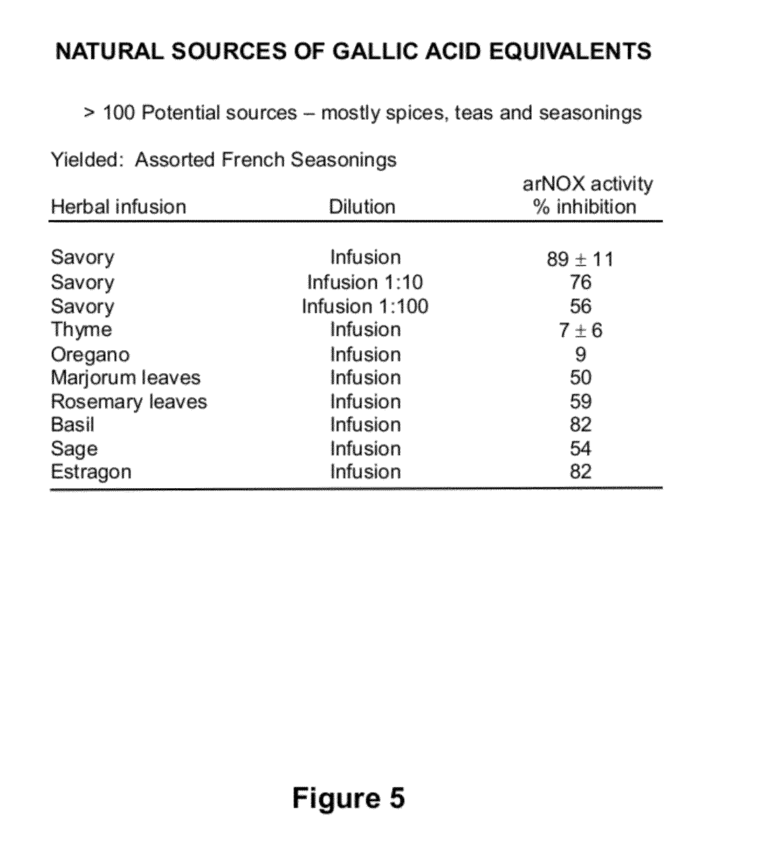

Structure-function analyses were carried out based on competitive inhibitors of xanthine oxidase. These compounds serve as competitive inhibitors of arNOX (FIG. 1). Among the competitive inhibitors identified were salicin, inositol, phytic acid, propolis and allopuranol (FIG. 2). All inhibited arNOX to varying degrees. Further structure-function analyses were carried out as shown in FIG. 3. An additional fifty compounds were identified with arNOX inhibitory activity more than half of which were derivatives of benzoic acid as exemplified by gallic acid (FIG. 4). Natural sources enriched in gallic acid or gallic acid equivalents also were analyzed as hot water soluble infusions (FIG. 5). Active sources were at a concentration of 125 mg/ml boiling hot water.

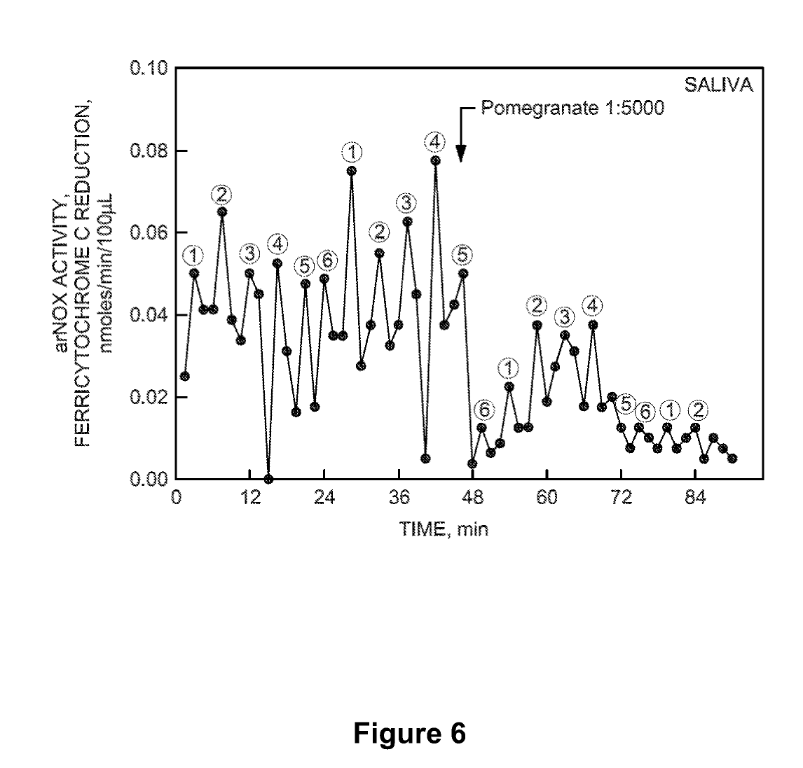

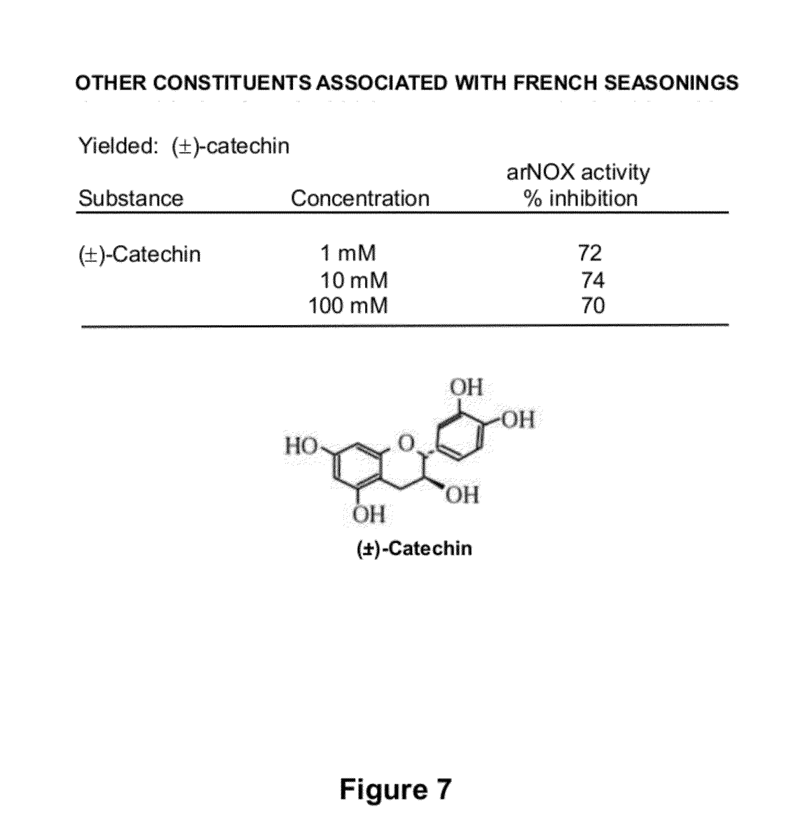

Predominantly culinary spices common in the French diet and known collectively as “Herbes de Provence” are rich sources of gallic acid equivalents and potent inhibitors of arNOX activity (FIG. 5). Also a rich source of gallic acid equivalents was pomegranate (FIG. 6). Another potent arNOX inhibitor associated with French seasoning was (±)-catechin (FIG. 7).

### EXAMPLE 3

### Identification of Active Ingredients

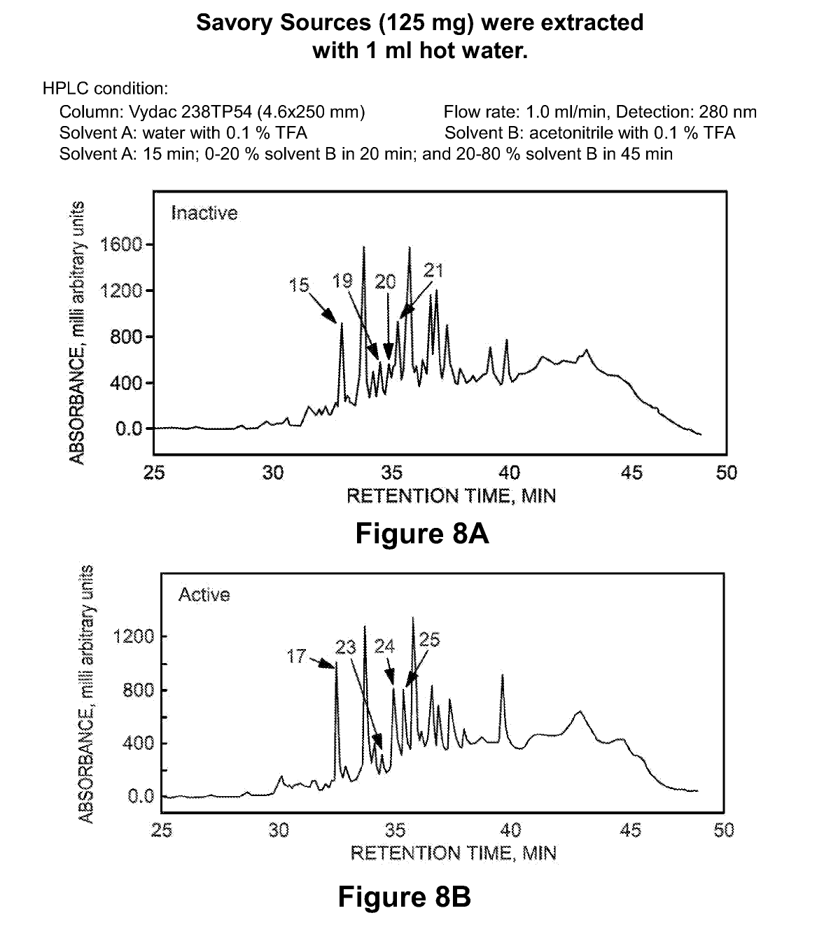

Infusions of two savory preparations obtained from the same provider (McCormicks) but with different expiration dates were analyzed by HPLC (FIG. 8). One (A) was inactive in inhibiting arNOX and the other (B) was active. Fraction 24 of B appeared to be present in greater abundance in the active savory preparation compared to fraction 20 in the inactive savory preparation to account for the arNOX inhibitory activity of the active preparation.

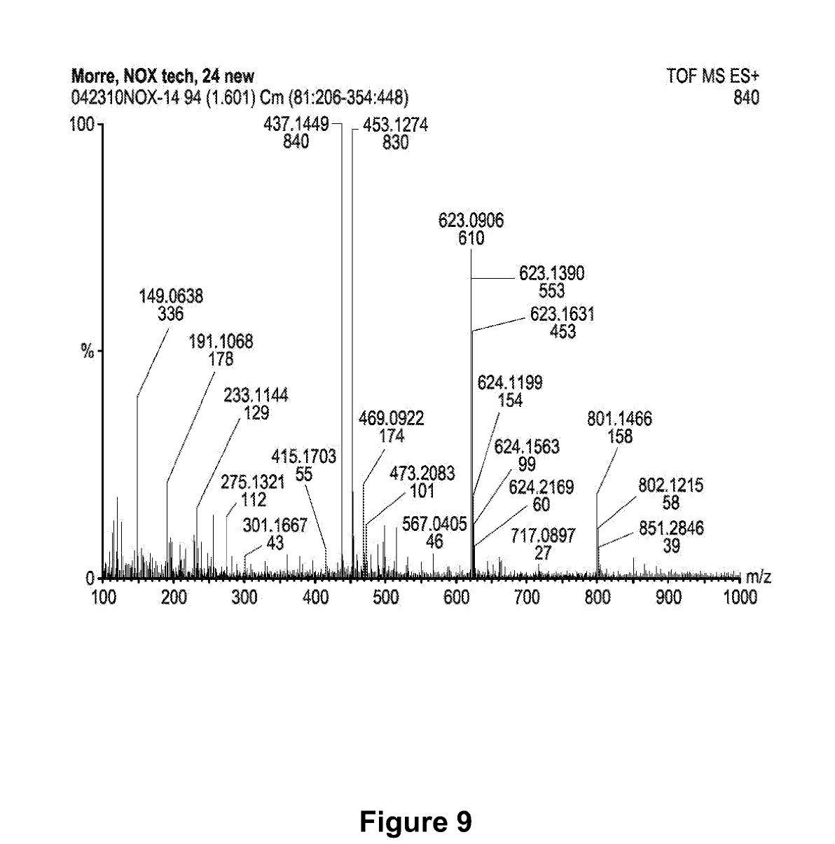

Difference mass spectrometric analysis of Fraction 24 (difference between Fraction 24 (active) and Fraction 20 (inactive)) was carried out (FIG. 9). Material at 623.0908 mass units decomposed to yield apigenin (270.9 mass units) as did the material at 453.1274 and the material at 437.1446 which differed by an —OH. These appeared to be water soluble derivatives of the flavonoid apigenin conjugated with sugars or hydroxy benzoic acid. The material at 149.0638 mass units was a dimethylated benzoic acid.

Infusions are prepared by adding 125 mg material (for herbs or spices, dry weight) to 1 ml boiling water. The solutions are allowed to cool slowly and steep at room temperature overnight. Pure compounds (gallic acid, apigenin, derivatives of benzoic acid) are prepared as 100 mM stock solutions in water, ethanol or DMSO, depending on solubility in a particular liquid.

Tinctures are prepared by adding 125 ml of material (fresh weight) to 1 ml ethanol and allowed to steep overnight (or from about 3-30 hours, about 6-24, or about 12-20 or about 18 hours). Insoluble and particulate material is allowed to sediment, and the soluble material is decanted. Tinctures, infusions and stock solutions of pure compounds are tested undiluted, diluted 1:10, 1:100 and 1:1000 to determine EC₅₀ (the dose which causes 50% inhibition of arNOX activity). For tinctures or infusions, aliquots of 60 μl are added to 2.5 ml arNOX assay. For stock solutions (undiluted or diluted), aliquots of 2.5 μl are added to 2.5 ml arNOX assay. For example, addition of an undiluted 100 mM stock solution results in a final concentration of 100 μM in the assay.

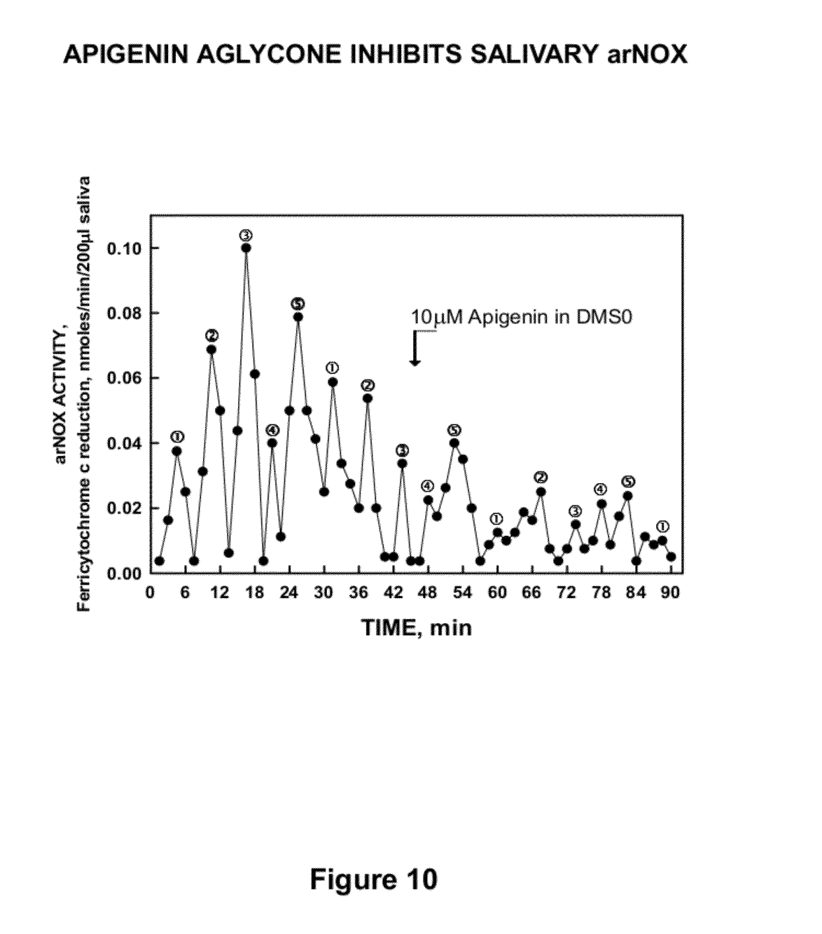

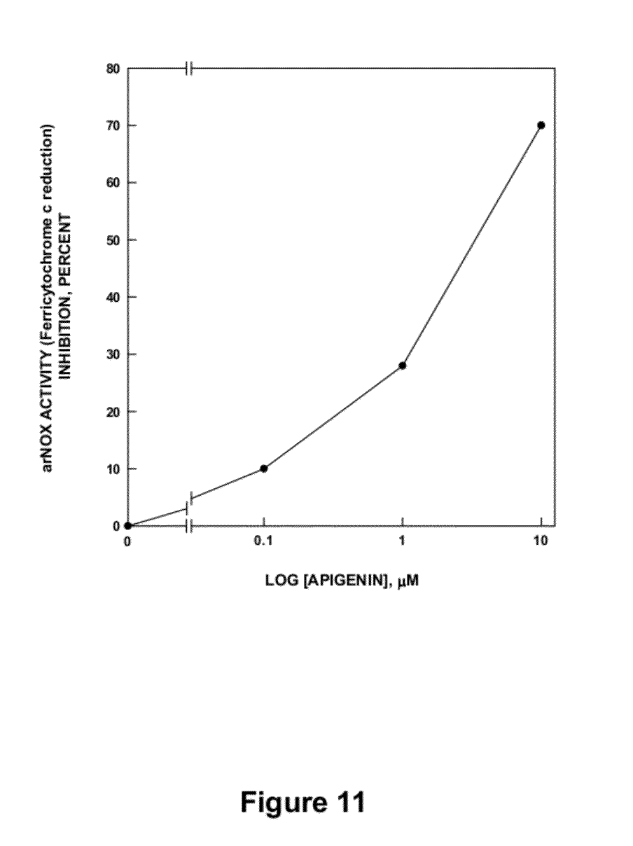

Apigenin aglycone supplied as a DMSO solution (insoluble in water) inhibited arNOX activity at 10 μM (FIG. 10) and the response was proportional to the logarithm of dose over the range 0.1 to 10 μM (FIG. 11). DMSO alone has no effect on arNOX activity.

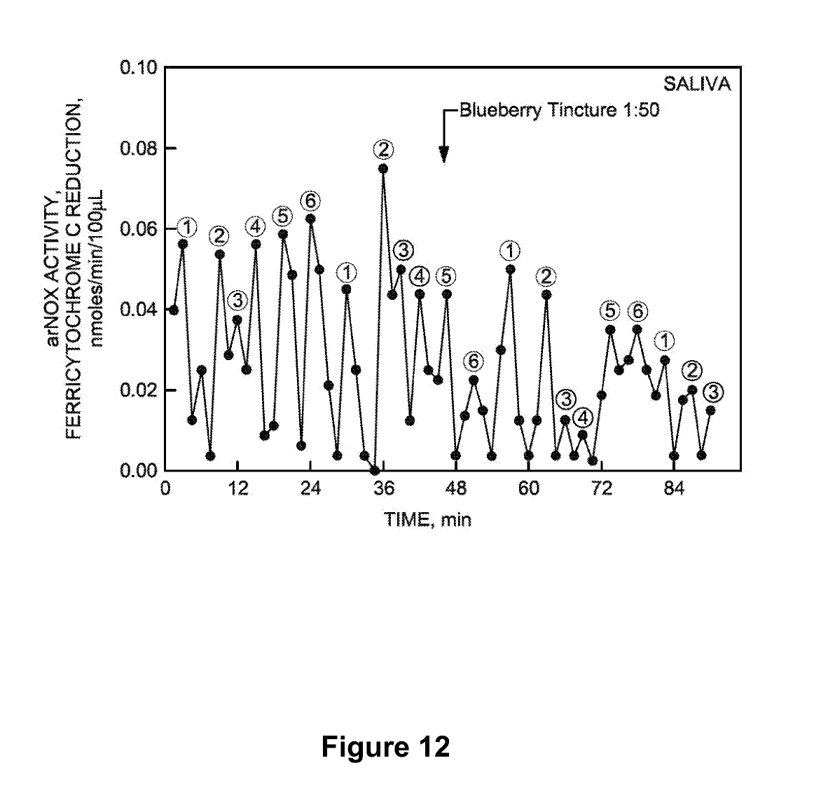

Hot water extracts of the majority of natural sources highly enriched in apigenin such as chamomile tea or dried parsley were inactive because of the low water solubility of apigenin. Tinctures (ethanol extracts) were prepared to inhibit arNOX from these sources. Similar results were obtained for blueberry (FIG. 12) and other blue fruits. Blueberries or other blue fruits, fresh or frozen, are extracted with ethanol (125 mg/ml) to prepare a tincture which was assayed directly (60 μg/2.5 ml) or assay mixture. Ethanol alone was without effect.

These findings suggest that the unique arNOX inhibitory activity of the apigenin and other flavones of the French seasonings are due to their presence as the water soluble conjugates revealed by mass spectroscopy (FIG. 9).

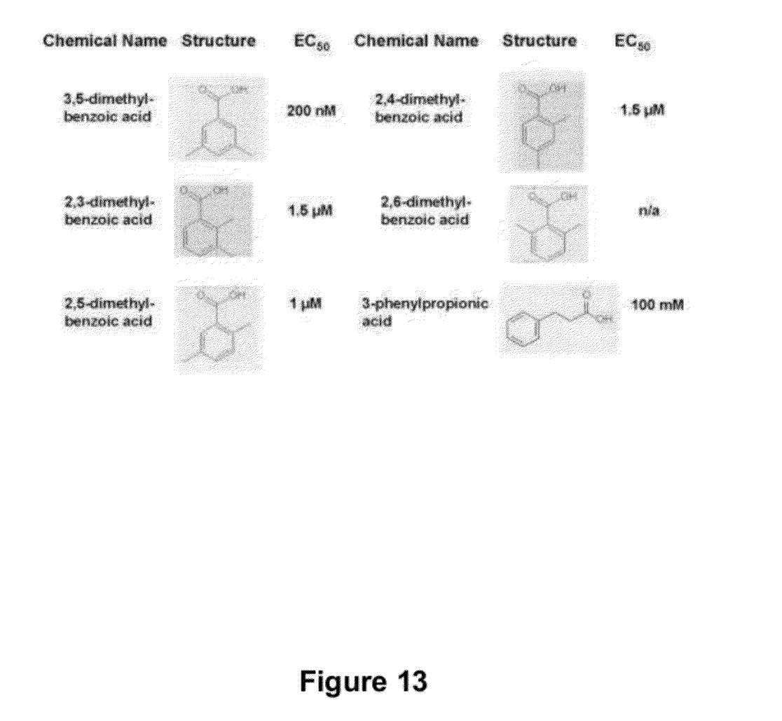

The material at 149.0638 mass units from the active savory fraction was dimethylated benzoic acid (FIG. 9). Five dimethylated benzoic acids were tested for arNOX inhibitory activity. All were inhibitory (FIG. 13). 3,5-dimethylbenzoic acid was the most inhibitory, with an EC₅₀ of 200 nM.

### EXAMPLE 4

### Kinetics of arNOX Inhibition

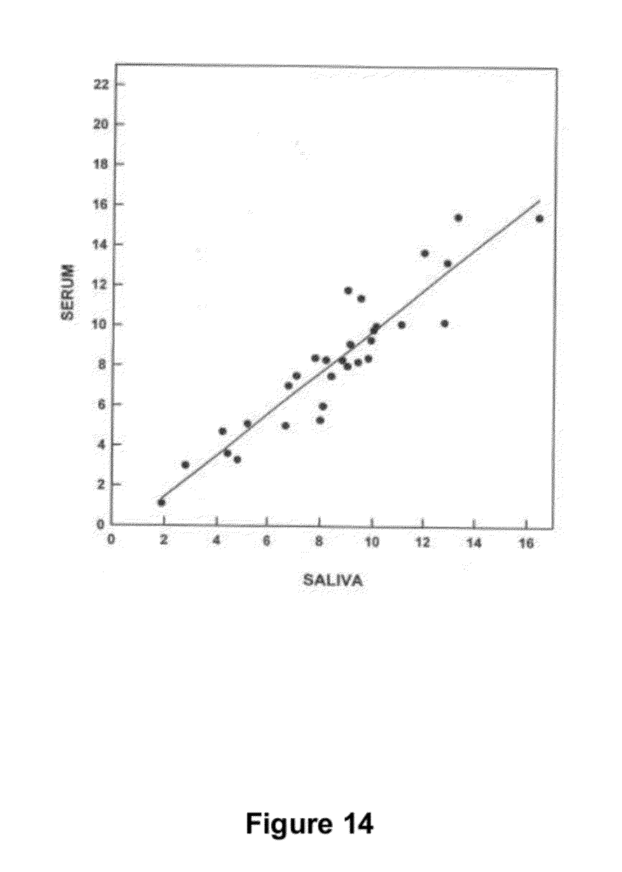

To determine the kinetics of arNOX inhibition, capsules containing 350 to 700 mg of selected seasonings and active ingredients were administered orally to a male volunteer and the arNOX activity of collected saliva was analyzed serially. arNOX activity of saliva and sera correlate closely (FIG. 14) such that salivary arNOX is reflective of the inhibition of serum arNOX.

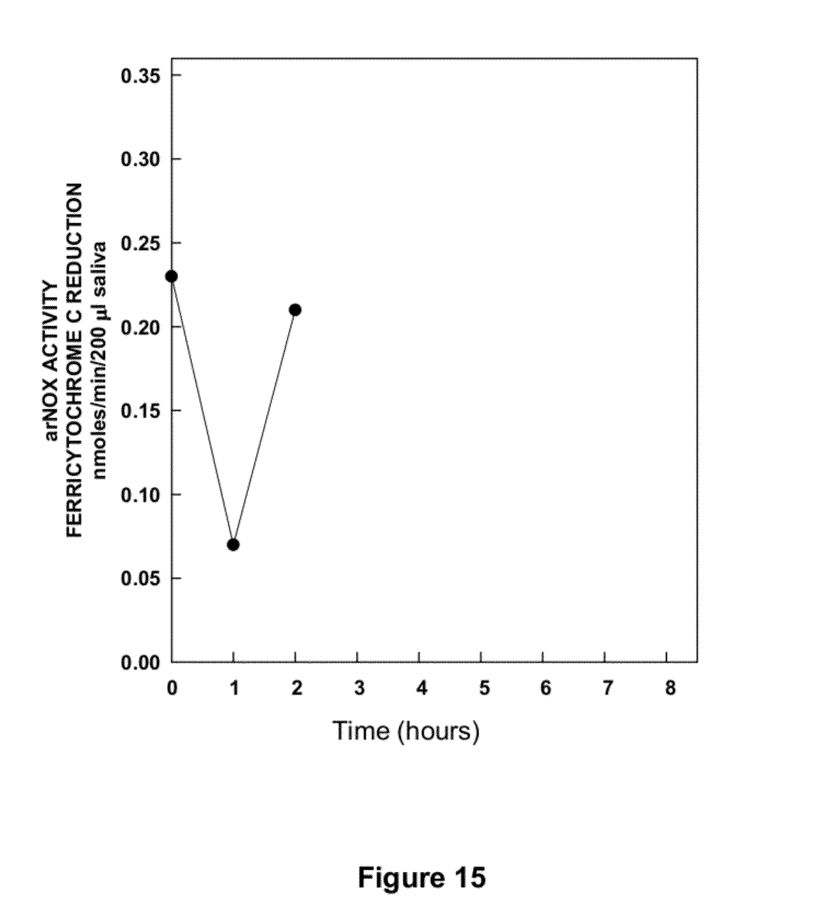

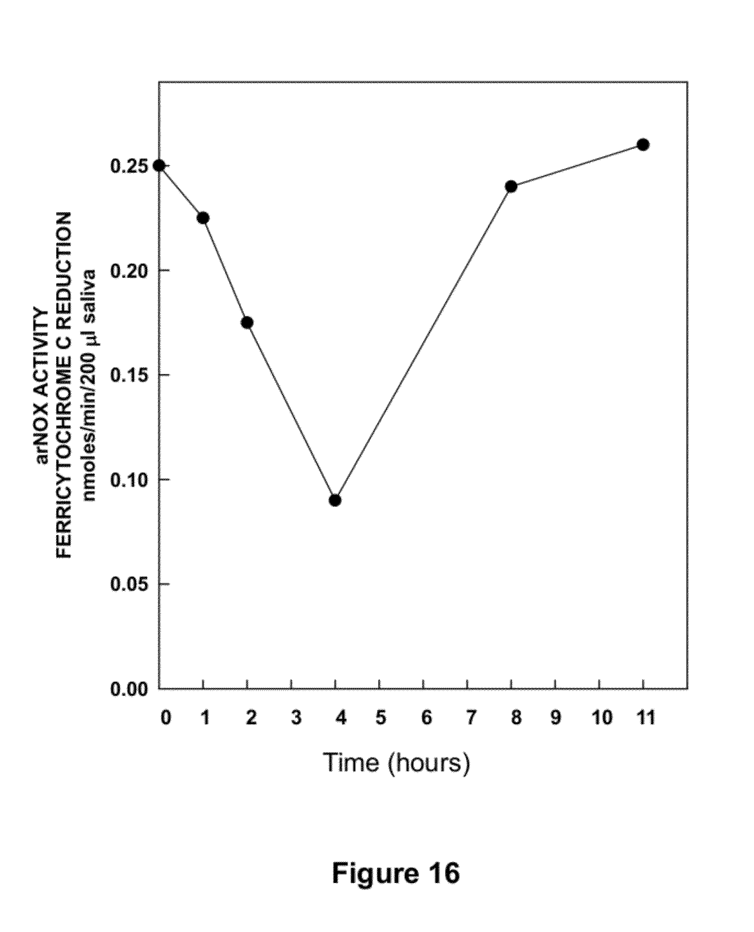

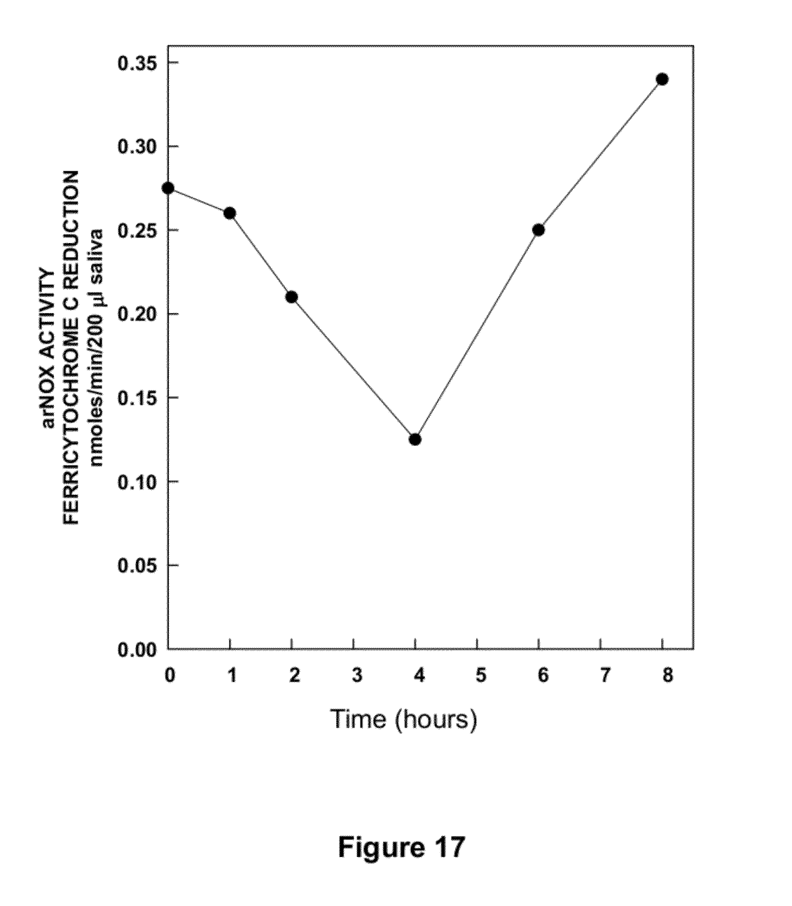

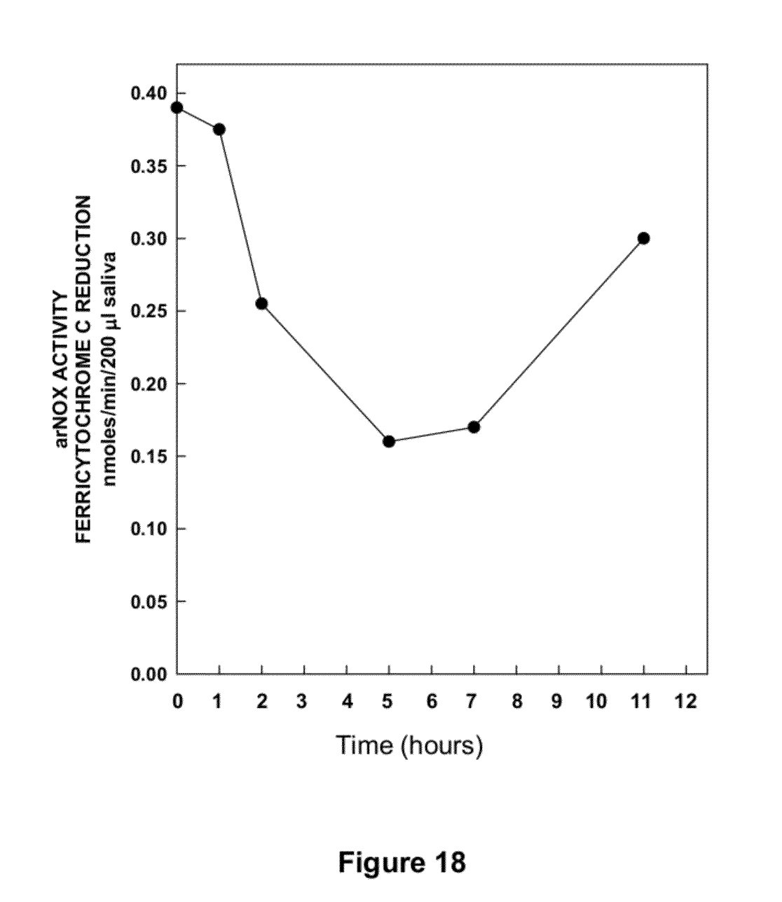

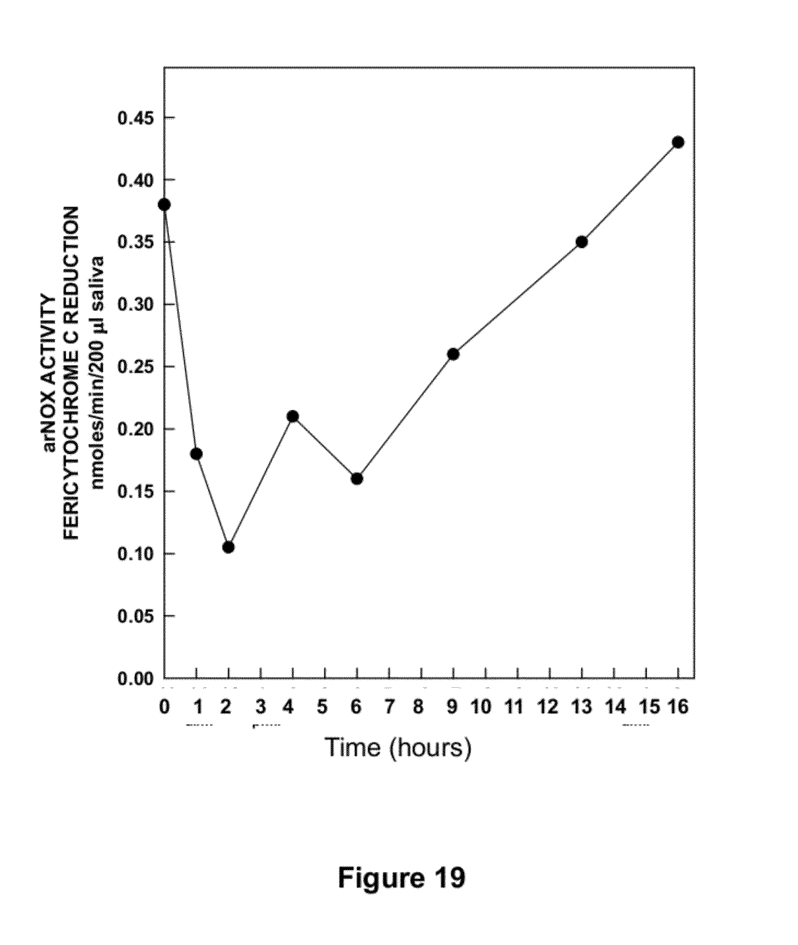

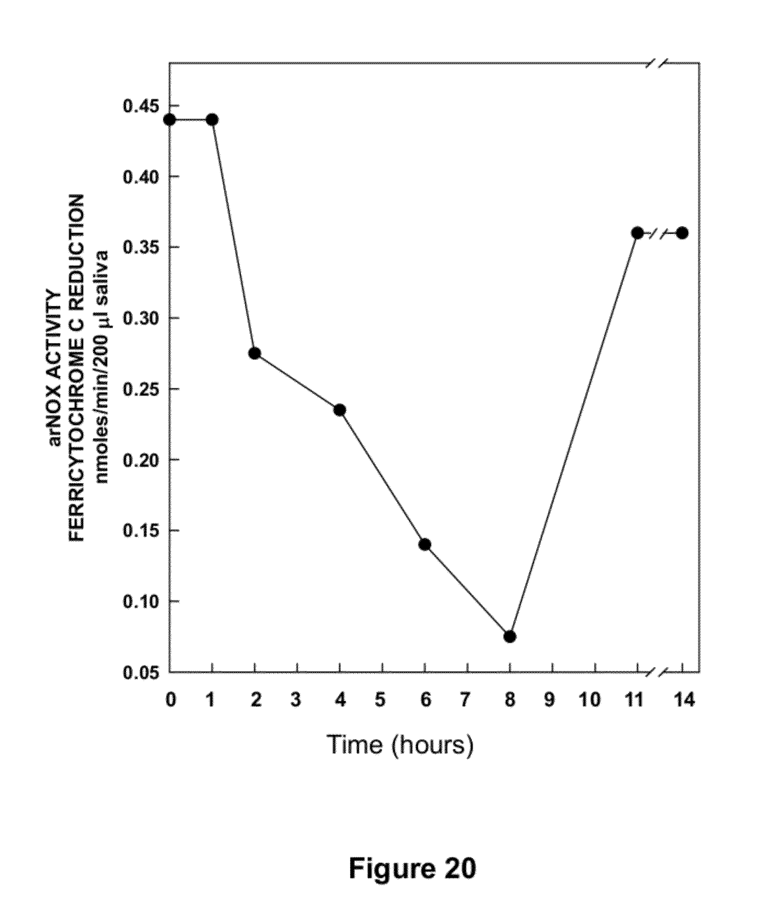

Each arNOX inhibitor source exhibited its own characteristic time course of inhibition (FIGS. 15 to 20). Inhibition by savory leaf was rapid and of short duration. The maximum inhibition of 70% was observed after 1 h followed almost immediately by a return to base line. French Tarragon (Estragon) leaf was similar to savory leaf in its kinetic properties. Maximum inhibition was delayed with domestic tarragon until about 4 h with return to baseline by 8 h (FIG. 16). Interestingly, finely ground savory gave kinetics different from savory leaf with maximal inhibition between 4 and 6 h similar to domestic tarragon and a return to baseline between 9 and 12 h (FIGS. 17 and 18). When savory leaf and ground savory were combined in a single oral dose, the inhibition kinetics reflected both sources with one maximum after 2 h corresponding to savory leaf and another maximum after 6 h corresponding to finely ground savory (FIG. 19). Return to baseline was not until after 16 h. The greatest delay in inhibition was that observed with gallic acid where maximum inhibition occurred slowly over 8 h with maximum inhibition (84%) seen 8 h after administration (FIG. 20). Return to baseline occurred between 11 and 14 h with gallic acid.

The different kinetics exhibited by the different natural sources of arNOX inhibitors offered the opportunity of preparing combinations of inhibitor sources to achieve broad spectrum inhibition as observed in FIG. 19 for the two dosage forms of savory. Currently a mixture of savory leaf, ground savory and gallic acid would be expected to be sufficient to offer 8 h overnight protection with 24 h protection offered by 3 capsules per day. By incorporating different sustained release agents, we anticipate 12 h protection from a two capsule/day regimen with the possibility of extending the regimen to a one capsule/day 24 h protection regimen. This is one of several aspects of the present compositions and methods that is unique and makes possible a preventive or therapeutic utility of the technology of importance to treating aging-related damage in individuals as they age beyond 30 years.

### EXAMPLE 5

### LDL Oxidation

Levels of oxidized LDL in serum were measured by using a malondialdehyde protocol modified from Smith et al. (Smith et al. 1976. J. Lab. Clin. Med. 88:167-172). Briefly, the sera were combined with a mixture of 20% (w/v) trichloroacetic acid and 0.6 M HCI containing 0.06 M thiobarbiturate and heated for 15 min at 100° C. Absorbance was measured at 532 nm from spectra obtained between 300 and 600 nm with malondialdehyde equivalents calculated for 1,1,3,3-tetramethoxypropane (Aldrich) standards.

Lipoproteins were isolated by flotation ultracentrifugation. Sera of healthy volunteers were collected using IRB-approved protocols. Informed consent was used. Plasma membranes were isolated from a plant source enriched in arNOX and compared to plasma membranes from a comparable plant source not enriched in arNOX. Plasma membranes were isolated by aqueous two phase partitioning as described (Morré D J and Morré D M. 1989. BioTechniques 7:946-958).

arNOX proteins are unique among the ECTO-NOX (ENOX) proteins in that they mediate the generation of superoxide at the cell surface and, as shed proteins, appear in the circulation and other body fluids (including saliva, urine, perspiration and interstitial fluids) (Morré D M, Guo F, Morré D J. 2003. Mol. Cell. Biochem. 254:101-109; Morré D J and Morré D M. 2003) Free Radical Res. 37:795-808; Morré D J and Morré D M. 2006. Rejuvenation Res. 9:231-236). The superoxide generated affords an opportunity to form H₂O₂ and other reactive oxygen species for propagation to adjacent cells and tissues and for direct oxidation of serum lipoprotein particles. Because arNOX is shed, the reactive oxygen species generated from the superoxide becomes accessible to lipoproteins in the circulation resulting in their oxidation and increased atherogenic risk as well as damaging to adjacent cells and extracellular supporting matrices that are important to skin health.

A further unique feature of the arNOX proteins is their apparent absence (or presence at levels below the limit of detection) for cells and sera of young individuals. They then increase with increasing age (>30 y) to ca. age 60-70 (Morré et al. 2009. BioFactors 34:237-244). The distribution of arNOX activity with age correlates closely with the American Heart Association's assessment of risk for coronary artery disease.

That the arNOX activity of plasma membranes is active in oxidizing lipoproteins was demonstrated from data which show malondialdehyde-like materials formed during 2 h of incubation of lipoprotein particles isolated from human sera with plasma membranes expressing high levels of arNOX compared to plasma membranes lacking arNOX (see FIG. 5). The amount of lipoprotein oxidation that occurred during the 2 h of incubation was enhanced 13 fold by the presence of the plasma membranes expressing arNOX compared to plasma membranes lacking arNOX. Similar results have been obtained subsequently using various soluble (or recombinant) arNOX sources.

This work has subsequently advanced at NOX Technologies, Inc. to result in a nutritional supplement based on use of the arNOX protein as the basis for inhibitor selection. Among the most effective inhibitors suitable for dietary intervention are certain dietary constituents known collectively as “Herbes de Provence”. Inhibitions by herbal infusions of savory, estragon (tarragon), basil, marjoram, rosemary or sage at a final concentration of 7.5 μg/mL in the assay varied from 50 to 90% along with a corresponding inhibition of arNOX-catalyzed lipid oxidation. Savory and estragon (tarragon) were effective at concentrations as low as 75 ng/mL in the assay. Also effective were gallic acid at a final concentration of 0.14 μM (10 ng/mL) and (±)-catechin at 100 μM (300 ng/mL).

Each herbal or phenolic arNOX inhibitor source exhibited its own characteristic time course of inhibition. The different kinetics exhibited by each of the various sources of arNOX inhibitors offers the opportunity of preparing combinations of inhibitor sources to achieve broad spectrum inhibition. However, by formulating the herbal preparations as sustained release preparations, 24 h protection was attained with just two 400 mg capsules/day (one in the morning and one before bedtime). It is this aspect that makes possible a therapeutic utility of the technology to reduce aging-related arterial damage from oxidized circulating lipoproteins in individuals as they age beyond 30 years.

Serum and salivary arNOX are highly correlated (r²=0.86, p<0.0008 in a study of 48 individuals in the age range 30 to 65 y; contracted research, NuSkin International, Provo, Utah). Hence, detailed kinetic data could be obtained from measurements of salivary arNOX. Similar studies to measure arNOX levels of sera in response to arNOX inhibitory supplements as proof-of-concept have been conducted.

All references cited herein are hereby incorporated by reference in their entireties to the extent they are not inconsistent with the present disclosure. These references reflect the level of skill in the relevant art. References cited herein indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

When a group of substituents or components is disclosed herein, it is understood that all individual members of those groups and all subgroups, including any isomers and enantiomers of the group members, and classes of compounds that can be formed using the substituents are disclosed separately. When a compound or component is claimed as part of a composition or method, it should be understood that compositions known in the art including the compounds or components disclosed in the references cited herein are not intended to be included. When a Markush group or other grouping is used herein, all individual members of the group and all combinations and subcombinations possible of the group are intended to be individually included in the disclosure.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individually or in any combination. One of ordinary skill in the art will appreciate that methods, device elements, starting materials, synthetic methods, and natural source material other than those specifically exemplified can be employed in the practice of the invention without resort to undue experimentation. All art-known functional equivalents, of any such methods, device elements, starting materials, synthetic methods, and natural source material are intended to be included in the present methods and compositions. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by particular embodiments and optional features, modification and variation of the concepts herein disclosed may be resorted to by those skilled in the art, and that such modifications and variations are considered to be within the scope of this invention as defined by the appended claims.

In general the terms and phrases used herein have their art-recognized meaning, which can be found by reference to standard texts, journal references and contexts known to those skilled in the art. The following definitions are provided to clarify their specific use in the context of the present methods and compositions.

One skilled in the art readily appreciates that the present invention is well adapted to carry out the objects and obtain the ends and advantages mentioned, as well as those inherent in the present therein. The methods, components and materials described herein as currently representative of particular embodiments are provided as examples and are not intended as limitations on the scope of the claimed invention. Changes therein and other uses which are encompassed within the spirit of the invention will occur to those skilled in the art, are included within the scope of the claims.

Although the description herein contains certain specific information and examples, these should not be construed as limiting the scope of the claimed invention, but as merely providing illustrations of some of the embodiments thereof. Thus, additional embodiments are within the scope of the invention and within the following claims.

It should be noted that the attending physician, cosmetician or other relevant practitioner knows how to and when to terminate, interrupt, or adjust administration due to toxicity, or to discomfort or other negative effects associated with the present methods and compositions. Conversely, the attending physician, cosmetician or other relevant practitioner also knows to adjust treatment to higher levels if the clinical response were not adequate (precluding toxicity). The magnitude of an administered dose in the management of the disorder of interest varies with the severity of the condition to be treated and to the route of administration. The severity of the condition may, for example, be evaluated, in part, by standard prognostic evaluation methods. Further, the dose and perhaps dose frequency, also varies according to the age, body weight, risk factors and response of the individual patient. A program comparable to that discussed above also may be used in veterinary medicine.

Depending on the specific conditions being treated and the targeting method selected, such agents may be formulated and administered orally, mucosally systemically, topically or locally. Techniques for formulation and administration are well known to the art. Suitable routes may include, for example, oral, rectal, transdermal, vaginal, transmucosal, or intestinal administration; parenteral delivery, including intramuscular, subcutaneous, intravenous, or intraperitoneal injections.

For injection, the agents of the invention may be formulated in aqueous solutions, preferably in physiologically compatible buffers such as Hanks' solution, Ringer's solution, or physiological saline buffer. For transmucosal or transdermal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular those formulated as solutions, may be administered parenterally, such as by intravenous injection. Appropriate compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes and then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present methods include compositions wherein the active ingredients are contained in an effective amount to achieve the intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions, including those formulated for delayed release or only to be released when the pharmaceutical reaches the small or large intestine.

The nutritional supplement, pharmaceutical and cosmeceutical compositions herein may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations and nutritional supplements for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical and nutritional supplement preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

What is claimed is:

1. A composition useful as a nutritional supplement or for topical application comprising an amount of at least one of an arNOX inhibitory agent and ar natural inhibitor source effective for ameliorating the effects of aging, wherein said natural source is at least one of basil (Ocimum basilicum), summer savory (Satureja hortensis), oregano (Oreganum vulgare) and thyme (Thymus vulgaris).

2. The composition of claim 1, wherein said composition further comprises one or more of lavender (Lavandula angustifolia), marjoram (Origanum majorana), rosemary (Rosmarinus officinalis), sage (Salvia officinalis) and/or fennel seed (Foeniculum vulgare), and/or tarragon (Artemisia dranunculus).

3. The composition of claim 1, wherein the inhibitory agent is a derivative of benzoic acid or a pharmaceutically acceptable salt thereof, dimethyl benzoic acid or a pharmaceutically acceptable salt thereof, apigenin, gallic acid or a pharmaceutically acceptable salt thereof, catechin, allopurinol, salicin, blueberry tincture, or pomegranate extract.

4. The composition of claim 1, wherein the inhibitory agent is a water soluble flavinoid derivative.

5. The composition of claim 1, wherein a natural inhibitor source is savory, estragon, tarragon, sage, basil, rosemary, marjoram or pomegranate alone or in combination with at least one additional arNOX inhibitor.

6. The composition of claim 1, wherein the composition further includes a cosmetically or pharmaceutically acceptable carrier or an ingestible carrier.

7. The composition of claim 1, wherein more than one arNOX inhibitory agent is present and wherein the more than one arNOX inhibitory agent is in the form of a plant powder, plant extract or plant infusion plant tincture or plant extract.

8. The composition of claim 7, wherein one arNOX inhibitor increases inhibition by the at least one additional arNOX inhibitory agents present therein.

9. The composition of claim 1, wherein the arNOX inhibitory agent is provided at a dose of between about 5 μg/ml to about 500 μg/ml.

10. The composition of claim 1, wherein the arNOX inhibitory agent is provided at a dose of between 10 mg and about 2000 mg of a combination of herbs and/or natural products per day for a human.

11. The composition of claim 1, wherein the arNOX inhibitory agent is provided at a dose of from 200 mg to about 600 mg of a combination of herbs and/or natural products per day for a human.

12. The composition of claim 1 comprising basil, 0-95%; thyme, 0-50%; oregano, 0-90%; tarragon, 0-95%; rosemary, 0-95%; lavender, 0-50%; sage, 0-95%; savory, 0-95%; marjoram, 0-95% (each weight/weight).

13. The composition of claim 12 comprising basil, 0-75%; thyme, 0-50%; oregano, 0-75%; tarragon, 0-75%; rosemary, 0-75%; lavender, 0-50%; sage, 0-75%; savory, 0-75%; marjoram, 0-75% (each weight/weight).

14. The composition of claim 12 comprising basil, 0-55%; thyme, 0-50%; oregano, 0-50%; tarragon, 0-50%; rosemary, 0-50%; lavender, 0-50%; sage, 0-50%; savory, 0-50%; marjoram, 0-50% (each weight/weight).

15. The composition of claim 1 formulated as a sustained release formulation.

16. The composition of claim 1 which is a nutritional supplement.

17. The composition of claim 1 which is formulated for topical application.

18. A method to inhibit the generation of reactive oxygen species by an aging-related isoform of NADH oxidase (arNOX), to ameliorate the effects of aging by administering an effective amount of a composition comprising at least one arNOX inhibitor of claim 1, to a in a human or animal in need thereof, whereby generation of reactive oxygen species by aging-related isoform of NADH oxidase, is inhibited and whereby an effect of aging is ameliorated.

19. The method of claim 18, wherein the composition is administered oraly as a gel capsule, pill or any other suitable oral dosage form or topically as a cream, milk lotion, gel or suspension.

20. The method of claim 18, wherein said arNOX inhibitory agents are administered at a frequency necessary to maintain said constant levels in a treated subject.

21. The method of claim 18, wherein the composition further comprises at least one arNOX inhibitor selected from the group consisting of gallic acid, apigenin conjugated with a sugar or hydroxybenzoic acid, apigenin aglycone, dimethylated benzoic acid, catechin, salicin.

22. A kit for dermal application useful in ameliorating the effects of aging comprising of at least one arNOX inhibitor and instructions for use, wherein the arNOX inhibitor is at least one of basil (Ocimum basilicum), summer savory (Satureja hortensis), oregano (Oreganum vulgare) and thyme (Thymus vulgaris).

23. The kit of claim 22, wherein the kit further comprises at least one arNOX inhibitory agent selected from the group consisting of lavender (Lavandula angustifolia), marjoram (Origanum majorana), rosemary (Rosmarinus officinalis), sage (Salvia officinalis), fennel seed (Foeniculum vulgare), and tarragon (Artemisia dranunculus).
